(19) United States
(12) Patent Application Publication
(10) Pub. No.: US 2003/0108962 A1
(43) Pub. Date: Jun. 12, 2003

(54) Method of cancer estimation by alp isozymes

(76) Inventor: Tsuneo Kobayashi, Tokyo (JP)

Correspondence Address: BROWDY AND NEIMARK, P.L.L.C., 624 NINTH STREET, NW, SUITE 300, WASHINGTON, DC, 20001-5303, US

(21) Appl. No.: 10/203,800

(22) PCT Filed: Dec. 7, 2001
(86) PCT No.: PCT/JP01/10736

(51) Int. Cl. G01N 33/574; G06F 19/00; G01N 33/48; G01N 33/50

(52) U.S. Cl. 435/7.23; 702/19

(57) ABSTRACT

By examining whether or not there occurs a minor cancer in the body and analyzing the ALP isozyme patterns for the total estimation, an early cancer not only in a specific organ but also in any site in the body is found out and a high-risk group is identified. Namely, a method of cancer estimation by ALP isozymes comprises the steps of: fractionating serum by electrophoresis; separating proteins in the serum; color-developing the ALP isozymes with an ALP isozyme-staining solution to thereby detect the ALP isozymes; and, estimating the risk of the cancer. The method is characterized in that the proliferation of cancer cells is totally analyzed based on, in the patterns of ALP I to ALP V isozymes: the appearance of the ALP I and the ratio thereof; the bioactivity ratio of ALP II to ALP III; and, changes in ALP isozyme angle (ALP-A) showing the sharpness of ALP II and ALP III, whereby the proliferation state of the minor cancer is estimated.

### FIELD OF THE INVENTION

The present invention relates to a method of early cancer finding-out and estimation by ALP isozymes, in which: the blood and urine of a cancer suspect is inspected to detect biochemically a minor cancer; and, the amount of the cancer is determined. More particularly, the present invention relates to the method which is capable of: detecting the minor cancer with the use of alkali phosphatase (ALP); and, precisely finding-out and estimating the risk of the cancer.

### BACKGROUND OF THE INVENTION

In an anticancer therapeutics, it is important to find out an early cancer and make the diagnosis thereof. Particularly, in the case where the early cancers are minor ones, there are often observed the instances of perfect natural recoveries. As a result, a great number of methods for finding out an early cancer in stomach, lungs, uterus, breast and like organs have heretofore been proposed and carried out.

Cancer cells often issue a special component not found in a normal tissue. Further, the amount of some component produced in a cancer suspect is often much more than that of the same component produced in a normal person. As described above, some characteristic components have been found in the cancer cells and called “tumor markers” which serves as “markers” for identifying the cancer cells.

Such tumor markers are as follows: “alphafetoprotein” in cancer cells in the hepar or liver; cancer cells such as “carcinoembryonic antigen” found in the stomach and large intestines; proteins produced only in embryos; a tumor-specific antigen which is capable of reacting on “sugar chain antigen 19-9” and found in cancer cells in the pancreas and like organs; “human chorionic gonadotron” found in cancer cells of uterus's villi; hormones such as “carcitonin” found in cancer cells of the dysparathyroidism and like hormones; “alkali phosphatase (herein after referred to as “ALP”); and, the like.

This ALP is one of important tumor markers, serves as an enzyme for separating phosphoric acid, and is found in the human body in different molecular weight in its organs such as placentas and small intestines, in addition to the above-mentioned bones and liver. In the case where a plurality of kinds of enzymes having the same function with respect to each of organs are found, they are called “isozymes”. For example, in patients suffering from bone cancers, liver cancers, multiple-myelomatosis and like cancers, they produce isozymes which are different in molecular weight from those produced in a normal person.

In a conventional method for examining the cancers with the use of such ALP isozymes, it is difficult to find out the cancers when the cancers do not sufficiently develop and therefore not reach a predetermined size. As a result, in the conventional method for finding-out and estimating the cancers with the use of the isozymes is poor in both “sensitivity” and “specificity”, wherein: the term “sensitivity” means a ratio in cancer of correct finding of the caners to failure of such finding as to the cancer patients; and, the term “specificity” means a ratio in cancer of correct finding of the normal persons to failure of such finding. Consequently, the conventional method for fining out the cancers with the use of the isozymes is used as a kind of auxiliary examinations in diagnosis of the cancers, or used as a kind of observatory examination in treating the cancers.

### TABLE I

Properties of Various-Types ALP Isozymes found in the Serumm:

### SUMMARY OF THE INVENTION

The present invention was made to solve the above problem. In other words, it is an object of the present invention to provide a method of cancer estimation using the ALP isozymes for finding out occurrence of any minor cancer in any part of a human body and estimating the risk of the cancers by analyzing in pattern the ALP isozymes to realize a total estimation of the cancers, wherein the early cancer occurring in a specific organ may be identified to enable a physician to prevent the cancer from developing.

The inventor of the present invention has noted a method for biochemically detecting the minor cancer with the use of a change in patterns of the ALP isozymes and a change/hour in each of Δ CEA and Δ ferritin, wherein the presence of the minor cancer is used as an index. In development of the cancers, which seems to be dedifferentiation or abnormal differentiation, the ALP play an important role. There is an intimate relationship between the proliferation of the cancer cells and the patterns of the isozymes ranging from ALP I to ALP IV in a normal range of the ALP's total bioactivity in the serum. Due to this, by analyzing a change (Δ) in value of the tumor marker for a period of a predetermined time and analyzing the isozymes, it is possible to detect the presence of any minor cancer having cancer cells the number of which is from 10⁴ to 10⁹ (i.e., in weight, from 10 microgram to 1 gram). In other words, the inventor of the present invention has found a novel method of cancer estimation by analyzing a change in pattern of the ALP isozymes for finding out occurrence of the minor cancer.

According to the present invention, a method of cancer estimation using ALP isozymes for finding out occurrence of a minor cancer and estimating the risk of the cancer through a process comprises the steps of: applying a blood serum to a support element having been immersed in a buffer solution; fractioning the support element at a predetermined liquid temperature through an electrophoresis; isolating proteins from the serum; color-developing the proteins with the use of an ALP isozyme-staining solution to detect the ALP isozymes; and, estimating the risk of the cancer, characterized in that:

proliferation of cells of the cancer is totally analyzed by using values of APA and APT, wherein the values are determined with reference to patterns of ALP I to ALP V isozymes on the basis of the following parameters: occurrence of ALP I and a ratio of the occurrence in the ALP isozymes; a ratio of ALP II to ALP III in bioactivity in the ALP isozymes; and, a change in ALP isozyme angle (ALP-A) in the ALP isozymes, the change showing both ALP II and ALP III in sharpness in the ALP isozymes;

whereby the proliferation state of the minor cancer is estimated.

In the above method of the present invention, the APA obtained from the ALP isozymes is represented by the following equation:

APA=α/θ1,

where θ1 is an angle formed between: a tangential line appearing in the anode side of the ALP II; and, a line extending between a peak point of the ALP II to and a peak point of the ALP III, and the α is a distance between an intersection of these two tangential lines and the peak point of the ALP III, and

in a condition in which ALP IV occurs, the APA is represented by the following equation:

APA=β/θ2,

where β is a distance (cm) between: an intersection with the tangential line of the ALP II and a peak point of each of the ALP III and the ALP IV; and

a value of the APT is determined by dividing an overlapping line (γ cm) by a dividing line (δ cm), wherein the overlapping line (γ cm) extends from the ALP II to a change point where the ALP II changes into the ALP III; and, the dividing line (δ cm) is obtained in a position lower than the peak point of the ALP II by 1 cm.

In the above method of the present invention, since there is an intimate relationship between the proliferation of the cancer cells and each of the APA and ALT, it is possible to detect the minor cancer the number of cells of which is from 10⁴ to 10⁹ by analyzing in pattern these ALP isozymes. In other words, the ALP I occurs when the total bioactivity found in the cancer cells of protopathic liver cancers, liver immersion oils, congested livers and also found in adipohepatic, cells increases. This makes it possible to predict the development of each of these cancers. The ALP II is called “hepatogenous ALP”, and found also in cancerous hydropericardiums. When the total bioactivity of the ALP III increases, it is known that such total bioactivity increases in any one of the osteoglast's new generation, the hepatocirrhosis and the chronic kidney failure. In the case where the ALP is within a normal range of values, the ALP III often increases in a certain period of a new generation of tissues, for example, during a period of proliferation of the minor cancer, a period of regeneration of a liver, and a period of slow-development of clinical cancers.

Further, when the clinical cancers slow down in development, the ALP III transforms into the ALP II due to the presence of neuramidinase in the blood so that the amount of the ALP III decreases. There is a strong probability that the ALP III is a modification enzyme capable of realizing a so-called “dephosphorylation” associated with the proliferation of new-generated cells.

It is possible for the method of the present invention to precisely identify each of the ALP II, ALP III and the ALP IV through analysis of reconstructed patterns in the patterns of each of the ALP isozymes ranging from the ALP I to the ALP IV.

It is possible for the method of the present invention to precisely and further efficiently identify each of the ALP II, ALP III and the ALP IV through analysis of reconstructed patterns in the patterns of each of the ALP isozymes ranging from the ALP I to the ALP IV. Particularly, heretofore, in the conventional method, though the ALP IV is detected much more in ratio than the other isozymes in the cancer tissues, the occurrence of the ALP IV seems to be within a range of from 1% to 3%. Due to this, the ALP IV seems to be low in bioactivity, and therefore often overlooked or mixed with the ALP III. However, it is possible for the method of the present invention to efficiently detect such hard-to-find ALP IV through analysis of the reconstructed patterns above described.

Further, in the method of the present invention, the proliferation state of the cancer's cells is analyzed using changes in each of the patterns of the ALP isozymes. A tumor is classified in its proliferation level into the following five stages by using a tumor marker: a stage I which is an ideal condition free from any minor cancer; a stage II corresponding to a precancerosis measured in microgram unit; a stage III corresponding to a precancerosis measured in milligram unit; a stage IV corresponding to a so-called “precancerosis”; and, a stage V in which the amount of cancers is equal to or more than 1 gram. A change of the tumor marker with elapsed time is analyzed in combination with the analyzation of the cancer's cells in its proliferation state. As a result, it is possible to judge the presence of the minor cancer and its proliferation state.

In the estimation method of the present invention, in a condition in which the ALP isozymes are not precisely identified in their patterns, the tumor is classified in its development level on the basis of the tumor markers. By introducing these classification models in development level, it is possible for the estimation method of the present invention to precisely estimate the cancers ranging from the minor cancers to the clinical cancers.

### BRIEF DESCRIPTION OF THE DRAWING

FIG. 1 shows graphs illustrating the patterns of the ALP isozymes ranging from the ALP I to the ALP IV.

FIG. 2 shows graphs and tables each of which illustrates the patterns of the ALP isozymes ranging from the ALP I to the ALP IV, wherein:

(a) illustrates the patterns of the ALP isozymes found in a 60-year-old male person before and after an operation of the person in his rectum cancers (in the electrophoresis of the ALP, a kit and a densitometer employing a film made of cellulose acetate “Titan III” produced by Helena Institute); and,

(b) illustrates the zymogram of the ALP found in a 56-year-old male patient suffering from his esophagus cancers.

FIG. 3 shows graphs and tables both of which illustrate the patterns of the ALP isozymes ranging from the ALP I to the ALP IV, wherein:

(a) illustrates variations in each of the marker and the isozymes found in a 33-year-old female person before and after a simple excision operation of the person in her breast; and,

(b) illustrates the isozymes found in the same person before and after an additional operation of the person.

FIG. 4 shows graphs illustrating the pattern of each of the ALP isozymes ranging from the ALP I to the ALP IV in the heat-treatment and the reconstruction experiment of each of the isozymes.

FIG. 5 shows diagrams, wherein:

(a) illustrates a change of the tumor markers with elapse time found in the serum of a patient (45-year-old, female) suffering from a IV-stage gallbladder cancers; and,

(b) illustrates an example in which a change of the Δ ferritin is classified into three types, i.e., an increasing type, an unchanged type and a down type.

FIG. 6 shows graphs, wherein:

(a) illustrates the Δ ferritin type; and,

(b) illustrates the increasing type.

FIG. 7 shows a diagram illustrating each stages in development of the cancers and a 5-stage estimation method using the ALP isozymes.

### BEST MODE FOR CARRYING OUT THE INVENTION

Hereinbelow, embodiments of a method of the present invention of cancer estimation using the ALP isozymes will be described with reference to the accompanying drawings.

The method of the present invention of cancer estimation using the ALP isozymes uses a change in patterns of important alkali phosphatase (ALP) isozymes and a change in a predetermined period of time in each of ΔCEA and Δ ferritin to detect biochemically a minor cancer (including some induced tumor markers), whereby the thus detected cancer is estimated. The ALP is found popularly in any parts of a human body. The bioactivity of the ALP increases in any one of the following cases: energy metabolism, differentiation in both leucocytes and mammogenesis; porosis; and, connective tissues under regeneration. Due to this, it is considered that the ALP plays an important role in cancerization of the tissues, wherein the cancerization is seen as a kind of dedifferentiation or abnormal differentiation. In other words, in a condition in which the total bioactivity of the ALP in the serum is within a normal range of value, there is an intimate relationship between the proliferation of the cancer cells and the patterns of the isozymes ranging from the ALP I to the ALP IV. For example, it seems to the inventor of the present invention that the number of cells of 10⁶ is a biological limit (reversible stage). In the present invention, any minor cancer having cancer cells the number of which is in a range of from 10⁴ to 10⁹ (i.e., in weight, from 10 microgram to 1 gram) can be detected through analysis of both a change (Δ) of the tumor marker in a predetermined period of time and the isozymes. The minor cancer is considered to be in a reversible stage. As for the clinical cancers which have a weight of equal to or more than 1 Kg, these clinical cancers are considered to be in unreversible stages.

Detectable ones of the minor cancers are classified into: micro-cancers, which are in the order of micrograms and have cancer cells the number of which is in a range of from 10⁴ to 10⁶; and, milli-cancers, which are in the order of milligrams and have cancer cells the number of which is in a range of from 10⁶ to 10⁹.

The method of the present invention of cancer estimation using the ALP isozymes finds out any occurrence of a minor cancer, and estimates the risk of the cancer through a process comprising the steps of: applying a blood serum to a support element having been immersed in a buffer solution; fractioning the support element at a predetermined liquid temperature through an electrophoresis; isolating proteins from the serum; color-developing the proteins with the use of an ALP isozyme-staining solution to detect the ALP isozymes; and, estimating the risk of the cancer

The method of the present invention is characterized in that:

proliferation of cells of the cancer is totally analyzed by using values of APA and APT, wherein the values are determined with reference to patterns of ALP I to ALP V isozymes on the basis of the following parameters: occurrence of ALP I and a ratio of the occurrence in the ALP isozymes; a ratio of ALP II to ALP III in bioactivity in the ALP isozymes; and, a change in ALP isozyme angle (ALP-A) in the ALP isozymes, the change showing both ALP II and ALP III in sharpness in the ALP isozymes, whereby the proliferation state of the minor cancer is estimated.

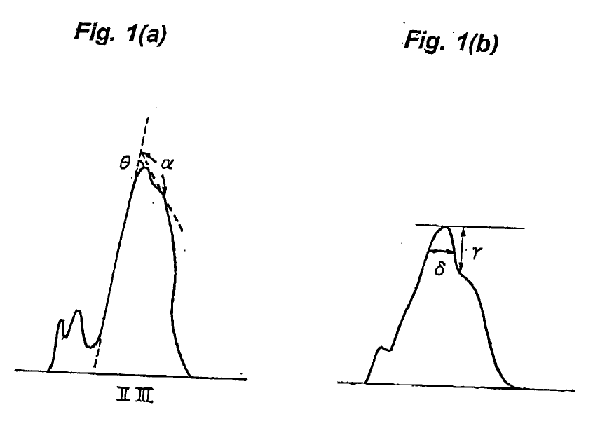

FIGS. 1 to 7 show the patterns of the ALP isozymes ranging from the ALP I and the ALP IV.

### ESTIMATION PROCESS

By analying a change in patterns of the ALP isozymes and a charge/hour in each of ΔCEA and Δ ferritin, it is totally judged as to whether or not the minor cancer occurs somewhere in a human body.

(1) ΔCEA:

The CEA was determined in quantity by using an enzymoimmunoelectrophoresis. Although the CEA remarkably varies among individual persons, it is well known that the CEA of a person varies in a range of equal to or less than 1.0 ng/ml for a long period of time. Here, a change in amount of the CEA is represented by the following equation:

ΔCEA=α₂−α₁

where:

α₁ is a value of the CEA at a predetermined time; and

α₂ is a value of the CEA at a time after the lapse of a certain period of time from the above predetermined time.

In this case, when the amount in variation of the ΔCEA is equal to or more than 0.4±0.1 ng/ml, such variation of the CEA is considered to be effective.

(2) A ferritin:

As for the ferritin, a hepatogenous ferritin was used in the embodiments of the present invention. In the embodiments, a change in amount of the ferritin is represented by the following equation:

Δferritin=β₂−β₁

where:

β₁ is a value of the ferritin at a predetermined time; and β₂ is a value of the ferritin at a time after the lapse of a certain period of time from the above predetermined time.

In this case, when the amount in variation of the Δferritin is equal to or more than 4±1 ng/ml, there is a probability of the presence of the minor cancer, provided that: in the case of anyone of substantial liver failures, substantial bladder failures and hemochromatosis, a value of the ferritin increases. On the other hand, since a value of the ferritin decreases in the case of hypoferremia, some consideration is required in estimation.

(3) Total estimation of the ALP isozymes:

The patterns of the ALP isozymes are totally judged based on the following factors:

1) occurrence of the ALP I and its ratio in occurrence;

2) a ratio of the ALP II to the ALP III in value of the bioactivity; and

3) the ALP isozyme angle APA, which represents a sharpness of each of the ALP II and the ALP III.

The angle APA is measured as follows (see FIG. 1): namely,

the APA is represented by the following equation:

APA=α/θ1,

where θ1 is an angle formed between: a tangential line appearing in the anode side of the ALP II; and, a line extending between a peak point of the ALP II to and a peak point of the ALP III, and the α is a distance between an intersection of these two tangential lines and the peak point of the ALP III, and

in a condition in which ALP IV occurs, the APA is represented by the following equation:

APA=β/θ2,

where β is a distance (cm) between: an intersection with the tangential line of the ALP II and a peak point of each of the ALP III and the ALP IV.

Next, examples of the method of the present invention of cancer estimation using the ALP isozymes will be described:

### EXAMPLE (1)

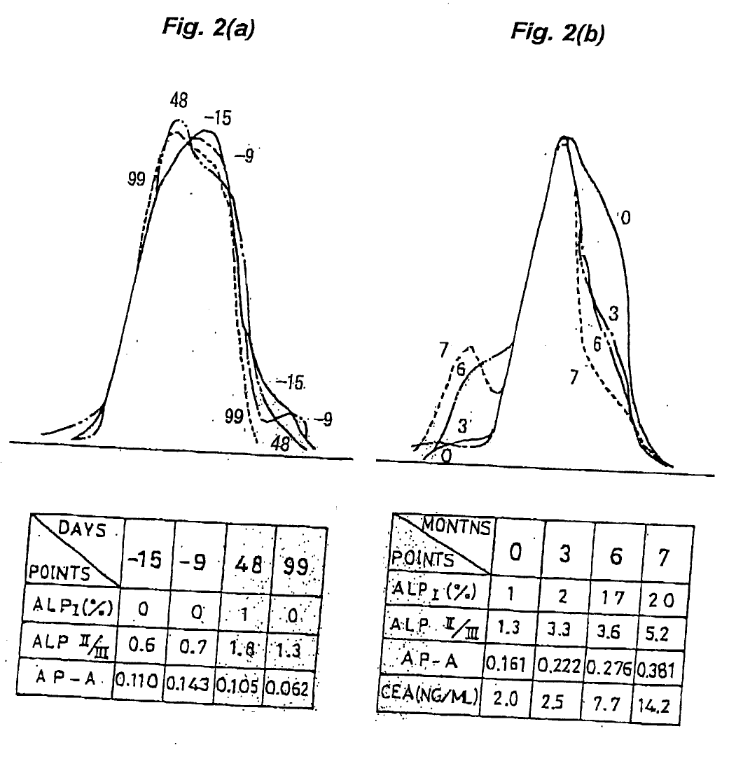

A 60-year-old male person had his rectum cancers (the I stage “pmN₀P₀H₀”) treated through a Miles' operation. The date of such operation was set at “zero”. Under such circumstances, the patterns of the ALP isozymes before and after the operation were analyzed according to the method of the total estimation (See FIG. 2(a)) As is clear from FIG. 2(a), an 1% of the ALP I still remained at a time after a 48-day lapse from the operation, and then disappeared at a time after a 99-day lapse from the operation. A ratio of the ALP II to the ALP III before the operation corresponded to reversed patterns of the ALP II and the ALP III, and had a value of equal to or less than 1.0. On the other hand, after the operation, the ALP decreased. And therefore, the value of the ratio after a 48-day lapse from the operation and the value of the ratio after a 99-day lapse from the operation increased to 1.8 and 1.3, respectively, within a normal range of values (1.6±0.4). Further, as to the APA, though the APA gradually became worse day by day, it was recognized that the APA recovered to be with in a normal range of values (equal to or less than 0.1±0.01) at a time after a 48-day lapse from the operation and also thereafter. Although several days were required before the APA recovered to be within a normal range, it is considered that the minor cancers, which remained in a peripheral area of the rectum cancers, required such a period of time before they disappeared.

### EXAMPLE (2)

FIG. 2(b) shows the ALP zymogram of a patient suffering from his esophagus cancers (which has metastasized to his liver). The total bioactivity of the ALP was within a normal range during the examination or estimation. The cancers gradually developed month by month. As a result, it was observed that a value of the CEA increased (FIG. 2(b). Also observed in gradual increase was each of the ALP I, a ratio of the ALP II/ALP III, and the APA.

### EXAMPLE (3)

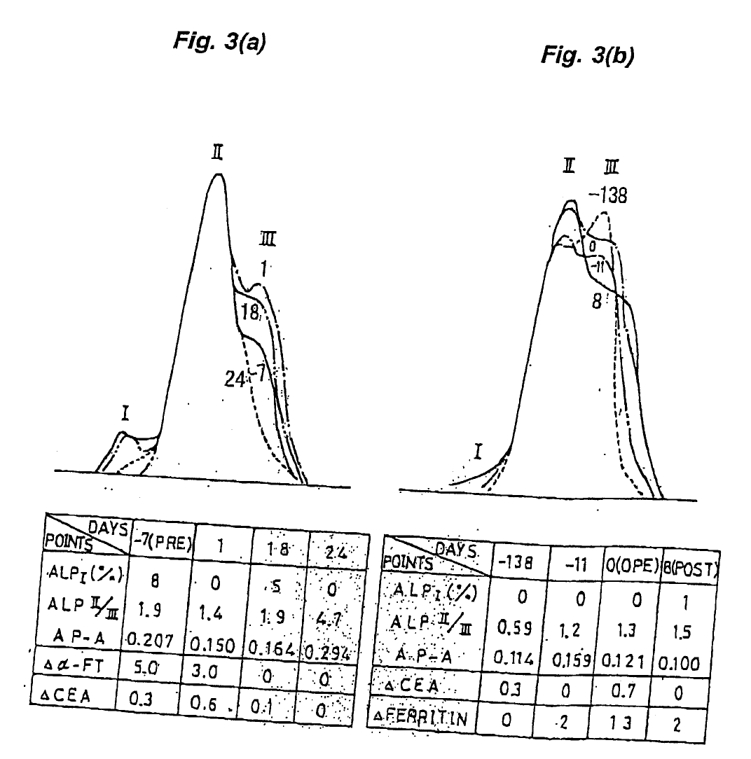

Variations in each of the markers and the ALP isozymes found in a 33-year-old female person before and after a simple excision operation of the person in her breast cancers (approximately 1 Kg) were examined (FIG. 3(a)). A value of the “Δ” in the tables corresponds to each of: a difference between the (α−FP) and a value of 2 ng/ml; and, a difference between the CEA and a value of 1 ng/ml (the minimum amount in the period of time). After the above excision operation, an ITC therapy was conducted twice on the same person. It is considered that FIG. 3(a) illustrates a post-gradually-growing process of one of the minor cancers after the operation, wherein the minor cancers were observed in a peripheral area of the cancer site having been operated. The ALP isozymes are speedy in reaction. Due to this, it is considered that each of the (α−FP) and the CEA seems to react with a slight time lag. Particularly, some characteristic patterns of the ALP III in decrease were well grasped numerically by using the APA. Although the APA decreased due to the operation, it was observed that the APA rapidly increased again with some time lag. At a time with the elapse of 24 days after the operation, the APA reached a value of 0.294, which indicated a probability of reoccurrence of the cancers.

The cancers developed again in the vicinities of the site of the same person having been operated after a 9-month elapse from the date of the operation, wherein the cancers were found to have a weight of approximately 0.5 gram. The thus developed cancers were removed by cutting. An oriental-medicinal-herb based therapy started seven days prior to the date of the operation. FIG. 3(b) shows the results of analysis of the ALP isozymes before and after the operation having been conducted again. Figures (arabic numerals) shown in FIG. 3(b) were the number of days having counted from the date of the operation, wherein the date of the operation was set at “zero”. In the drawing: the ΔCEA represents a residue resulted from an 1.7 ng/ml-reduction; and, the Δ ferritin represents a residue resulted from a 12 ng/ml-reduction. It is clear from the drawing that both the ferritin and the CEA increase up to a date immediately before the date of the operation.

Through analysis of the ALP isozymes in the serum found in the patient suffering from the cancers before and after the operation, it was suggested that there was an intimate relationship between: particularly, occurrence of the ALP I and a change in pattern each of the ALP I and the ALP III; and, the proliferation of the cancer cells. By checking a change of the tumor markers with elapsed time, and, further, by associating such check with the analysis of the ALP isozymes, it is considered that both the presence of the minor cancers and the proliferation of the cancer cells may be estimated.

Now, the analysis employed in the method of the present invention of cancer estimation using ALP isozymes will be described.

Under current circumstances in which each of the isozymes ranging from the ALP II to the ALP IV can't be precisely identified, in order to conduct an analysis in patters of these isozymes in a more precise manner, the following heat-treatment and reconstruction experiments of each of the isozymes were conducted:

### MATERIAL AND PROCESS IN EXPERIMENTS

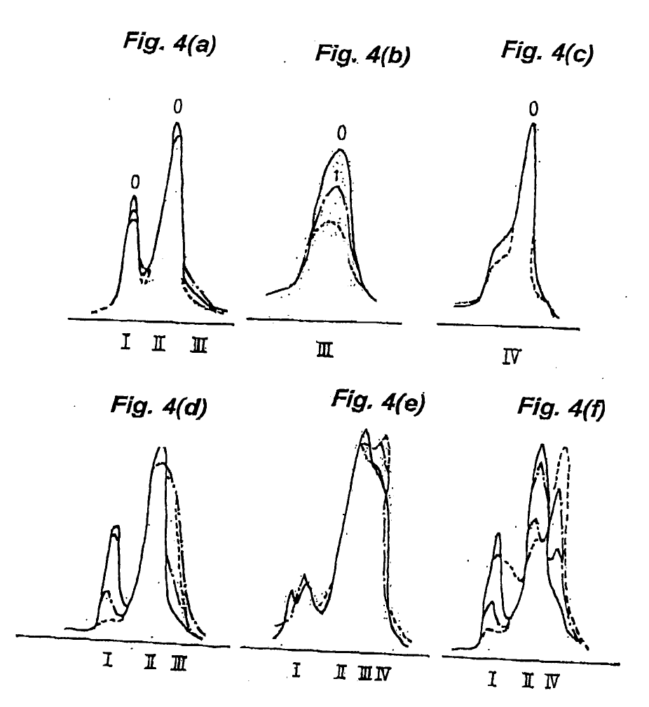

Used in the experiments were: a serum (hereinafter referred to as the “serum 2”) found in a cancer patient, wherein the serum 2 primary comprises the ALP II due to a remarkable reduction of the ALP III (See, FIG. 4(a), the reference numeral “0”); a serum (hereinafter referred to as the “serum 3”) found in a two-year-old preschool child, therein the serum 3 primary comprises the ALP III; and, a serum (hereinafter referred to as the “serum 4”) found in a pregnant person immediately after a delivery of her baby, wherein the serum 4 primary comprises the ALP IV.

### RESULTS OF EXPERIMENTS

Effects of the heat treatment on each of the serums:

FIGS. 4(a), 4(b) and 4(c) shows a series of zymograms obtained after a series of heat-treatments each performed on each of the serums for a period of from 0 to 5 minutes at a temperature of 56° C. (in the drawings, each of the reference numerals indicates a period of time in which the heat-treatment was performed). The drawings shows that: the ALP II is considerably stable at a temperature of 56° C. for a period of 5 minutes, and, a peak of the ALP II is sharp (See, FIG. 4(a)).

On the other hand, the drawing shows that: the ALP III has a smooth curve, which suggests the presence of a wider variation in molecular type of the ALP III. As for the ALP IV, the drawings shows that: the ALP IV is of a thermotolerant type; and, its sharp peak remarkably indicates that the ALP IV is originated in a late-stage placenta of the pregnant person (See, FIG. 4(c)).

In the reconstruction experiment 1 (FIG. 4(d)): the inventor of the present invention had noted the properties of the ALP II in this experiment, and analyzed the ALP II by electrophoresis. Through calculation of a bioactivity of each of the ALP II and the ALP III on the basis of the total bioactivity, a ratio of each of the bioactivity was determined. When the ALP III was gradually increased to the amount of each of 1, 25, 75 and 92 in percentage, the corresponding ALP II gradually reduced to the amount of each of 99, 75, 25 and 8 in percentage. In the vicinities of a peak in value of the ratio (ALP II/III), the number of fractions of the ALP III increases. Due to this, the graph of the ratio (ALP II/III) has a circular curve, which forms an obtuse angle in the vicinities of the above peak.

In a reconstruction experiments 2 (FIG. 4(e)), the presence of the ALP II was neglected in this experiment. The serum 2 and the serum 3 were mixed with each other to produced a mixture. Obtained from this mixture were the patterns of the isozymes comprising a 30% of the ALP II and a 70% of the ALP III, wherein the patterns are indicated by the reference numeral “0” in the drawings). These patterns were the same patters as those obtained in a normal person. In a condition in which a ratio of the serum 2 to the serum 3 was kept constant, the serum 4 was gradually added to the mixture of the serums 2 and 3 to produce another mixture which was then analyzed. As a ratio (%) of the ALP IV in the another mixture increased (in the drawings, see the reference numerals 8, 16 and 33), the graph became to have a shoulder portion which was then transformed into two peaks in pattern. In comparison with the reconstruction experiment 1 (FIG. 4(a)), it was possible to recognize the presence of the isozymes' patterns indicating the increase of the ALP IV in a condition in which: a sharp shoulder portion was formed in the peak of the ALP III; or, the two peaks appeared in the graph.

In the reconstruction experiment 3 (FIG. 4(f)): it was inspected about how the patterns of the ALP isozymes varied when the ratio of the ALP II to the ALP III changed. Each of The patterns of the ALP II to the ALP IV was transformed into a two-peak shape having a sharp and deep V-shaped form.

Through analysis of each of the ALP isozymes, it is possible to identify each of the ALP II, ALP III and the ALP IV in a simple manner. Since the cancers have a wide variation in molecular types, a detection rate of the ALP of a carchinoembryonic type is poor when a checking process of such ALP relies on a heat-treatment only. Heretofore, in spite of a high-detection rate of the ALP IV in the cancer tissues, it is said that an occurrence rate of the ALP IV in the blood of a patient suffering from the cancers is within a range of from 1 to 30 in percentage. As for the reason why a detection-rate of the ALP IV in the blood is poor, it is considered that: there is a probability of some connection with a leakage of the ALP IV into the blood; and, the presence of the ALP IV is overlooked due to its poor-bioactivity. Consequently, there is a possibility of an increase in a detection rate of the ALP IV when the patterns of the isozymes having been obtained through the above reconstruction experiments are utilized in detection. The ALP I occurs when the total bioactivity found in the cancer cells of protopathic liver cancers, liver immersion oils, congested livers and also found in adipohepatic cells increases. However, the inventor of the present invention has found out that: even when the ALP is within a normal range of values, the ALP I of the carchinoembryonic type often occurs; and, the ALP I increases as the cancers grow (FIG. 2(b)). The nearest one of the ALP I in characteristics is an ALP found in an embroyma in small intestine.

By the way, the ALP II is called “hepatogenous ALP”. However, in view of the fact that the ALP II is recognized also in a cancerous serous fluid in the pericardial cavity, the ALP II is considered to be a basic-type ALP.

It is well known that: in the case where the total bioactivity of the ALP increases, the ALP III increases in any one of the osteoglast's new generation, the hepatocirrhosis and the chronic kidney failure. When the ALP is within normal range of values, the ALP III increases in the new generation of cells, for example, in the proliferation of the minor cancers, in the regeneration of the liver, and in the slow-developing clinical cancers. When the slow-developing clinical cancers increase in developing speed, the ALP III decreases, probably, due to: the presence of neuramidinase in the blood; or, transformation of the ALP III into the ALP II. As for the ALP III, there is a strong probability of the ALP III being one of modification enzymes capable of realizing a so-called “dephosphorylation” associated with the proliferation of new-generated cells.

It becomes possible to estimate the presence of the minor cancers by diagnosis using the tumor markers, for example such as: the alkali phosphatase (ALP) isozymes found in the serum; the Δ CEA; and, the Δ ferritin; wherein each of the ΔCPA and the Δ ferritin is a difference measured in a predetermined period of time.

However, although it is said that an occurrence rate of the ALP IV in the serum is within a range of from 1 to 30 in percentage, the detection rate of the ALP IV in the cancerous tissues is considerably high. This is probably due to the presence of some mechanism, which prevents the tumor markers having been produced in the cancer cells from entering the blood. Consequently, in order to confirm the presence of a possible minor cancer, the inventor of the present invention, on the basis of the fact that vitamin A and hyperthermia are known effective in increasing the bioactivity of the tumor markers of cultivated cells in vitro, carried out an induction process of tumor markers originated in possible cancerous tissues for increasing the leakage of such possible markers, wherein the induction process was conducted by dosing vitamin A and also by hyperthermia by heating a deep area of a human body with the use of far infrared radiation which is effective in heating such a deep area.

Clinical Example

The total number of volunteers enrolled in a clinical example was 15, in which: the number of male persons was 4; and, that of female persons was 11. All the volunteers were sound in health and free from any cancer. Their ages were within a range of from 28 to 38.

As an example of the clinical cancers, gallbladder cancers found in a patient (45-year-old, female) were used. In induction process, vitamin A (retinal palmitate) was administrated at a dose of 50000 I.U. through intramuscular injection. Such intramuscular injection was followed by a rest period of a predetermined period of time. After the rest period, the patient was treated by hyperthermia at a temperature of 56° C. for a period of 20 minutes by heating the cancers with the use of far infrared radiation.

### RESULTS

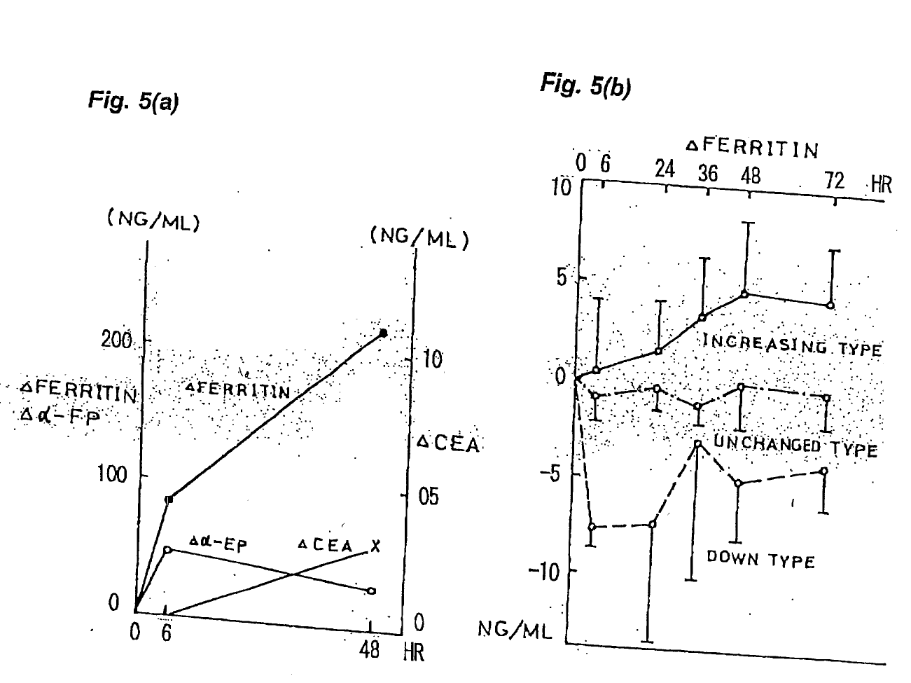

The patient suffering from the gallbladder cancers was treated by the induction process of the tumor markers found in the serum, wherein a change of the tumor markers with elapsed time was examined (FIG. 5(a)). As is clear from FIG. 5(a), with respect to each of the ferritin and the “α-FP”, a rapid leakage thereof into the blood was observed after a lapse of 6 hours. It was recognized that: the ferritin varied within a range of variation of 213 ng/ml; and, the “α-FP” varied within a range of variation of 50 ng/ml. As for the CEA, the CEA increased only by the amount of 0.3 ng/ml. Then, the induction process was applied to each of the volunteers the number of which is 15. At a time of each of 6 hours, 24 hours, 36 hours and 48 hours before the induction process, and, further, at a time of after a lapse of each of 6 hours, 24 hours, 36 hours and 48 hours from the induction process, a blood-gathering-operation was conducted on each of all the volunteers. Variations or differences in value of the ferritin found in the serums obtained through the above blood-gathering-operations at each of the above times were represented by the term “Δ ferritin”. A change of the Δ ferritin with elapsed time varied in mode. Due to this, the change of the Δ ferritin with elapsed time was classified in mode into the following three types: an increasing type; an unchanged type; and, a down type (FIG. (b)).

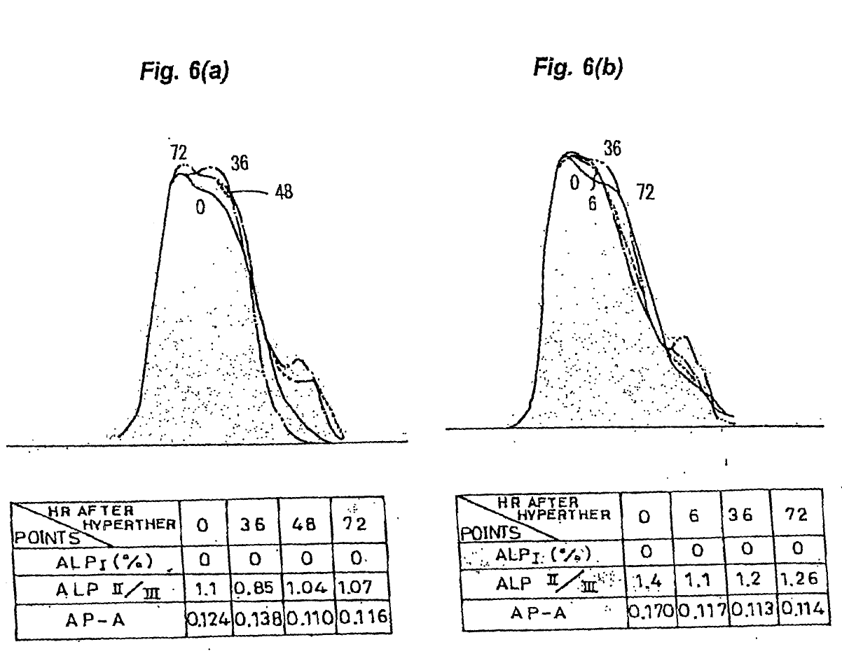

In the case of the unchanged type (n=4), the Δ ferritin varied in a width of equal to or less than 4 ng. In the case of the increasing type (n=6), it was observed that: the Δ ferritin began to increase after a lapse of 24 hours; and, then reached a peak after a lapse of 48 hours, in most cases. A width of variation of the Δ ferritin was within a range of from 7 ng to 12 ng. In the case of the down type (n=3), after a lapse of 6 hours from the induction process, it was recognized that the ferritin rapidly decreased (7.7 ng/ml±0.9 ng). A this time, a width of variation of the Δ ferritin was within a range of from 7 ng to 13 ng. After that, the width of variation of the Δ ferritin was recognized to gradually return to its initial value. Here, analysis in patterns of the ALP isozymes was conducted by using the method of the present invention. In the case of the unchanged type of the Δ ferritin, the bioactivity ratio of the ALP II ALP and the ALP III was also within a normal range, that is, within a range of from 1.6±0.4. Further, the APA was also equal to or less than 0.1, and found to be quite normal in value. In this unchanged type, the cancers was probably less in size than the minor cancer, and the number of its cells was probably equal to or less than 10⁴, which made it impossible for the cancers to be detected. Due to this, the patient was judged substantially normal. FIG. 6(a) illustrates the analysis of the ALP isozymes having been classified into the increasing type of the Δ ferritin, wherein each of the reference numerals indicates a lapse of times after the induction process.

As illustrated in the analysis shown in the drawings, a ratio of the ALP II to the ALP III after a lapse of 36 hours reached its abnormally low value (equal to or less than 1.0), and then gradually recovered. Such abnormal value was also observed as to the APA. Consequently, with respect to the Δ ferritin of the increasing type, the presence of these abnormal values wall suggest the presence of the minor cancer.

Lastly, FIG. 6(b) shows the analysis of the ALP isozymes with respect to the down type. In the case of the down type, a ratio of the ALP II to the ALP III after a lapse of 6 hours reached its abnormally low value, and then gradually recovered. Such abnormal value was also observed as to the APA. The APA was found to indicate values in the vicinities of the normal values after a lapse of 6 hours or more. On basis of the relationship in variation in time between the APA and the Δ ferritin, it is considered that the down type of the Δ ferritin probably indicates the presence of the minor cancer occurring with some inflammation.

As described above, even when the variation is minor, it is necessary to perform the induction process on the same patient. As a result, when the ΔCEA varies in a range of a value equal to or more than a value of (0.4±0.1) ng/ml and the Δ ferritin varies in a range of a value equal to or more than a range of from 4 ng/ml to 7 ng/ml, it is necessary to search the minor cancers. After that, the analysis of the ALP isozymes is performed to confirm the presence of the minor cancers. Particularly, when the thermotolerant ALP isozymes' fractions still remain alive in the patient body after the heat-treatment having been conducted at a temperature of 56° C. for a period of 10 minutes, the presence of such fractions must be checked. At the same time, the bioactivity of the thermotolerant ALP isozymes must be measured by carrying out a so-called “ultramicromeasurement” with respect to the bioactivity of the ALP isozymes (the heat-treatment conducted at a temperature of 65° C. for a period of 7 minutes) by using a fluorescence spectrophotometer. Combination of the above checking operation and the ultramicromeasurement makes it possible to detect the presence of the minor cancer without fail.

As for the clinical cancers: the Δ CEA varies in a range of a value equal to or more than (1.0±0.1) ng; and, the Δ ferritin varies in a range of a value equal to or more than 20 ng/ml.

Although only the induction patterns of the clinical cancers of the increasing type are shown in the above clinical example of the present invention, occurrence of the cancers of the down types was also very often observed. Each of the increasing type and the down type among the volunteers are considered to a minified one of each of the induction patterns of the clinical cancers. Further examination will be required in future to determine whether a difference in appearance between the down-type minor cancers and the increasing-type minor cancers is resulted from only the presence or absence of the inflammation or differences in existence condition among the cancers. Naturally, in the inflammation of any one of parenchymatous organs (in which inflammation a value of the ferritin increases) and the hemochromatosis, examination must be carefully conducted. At this time, analysis in patterns of LDH isozymes may provide important information and data. Further, since a value of the ferritin decreases when a hypoferremia is observed in the serum, it is necessary at first to cure the patient of his hypoferremia before the induction operation is conducted on the patient. Further, starvation often rapidly increases a value of the ferritin. In the case of a simple inflammation such as a hepatitis and the like, only a value of the ferritin rapidly decreases. No adverse effect on any one of the Δ CEA and the ALP isozymes can be recognized.

The Δ ferritin was classified in its change mode into three types, i.e., the increasing type in which a value of the ferritin increases; the down type in which a value of the ferritin decreases; and, the unchanged type in which a value of the ferritin remaines unchanged. When a value of the ferritin is within a range of from 4 ng/ml to 20 ng/ml and that of the Δ CEA is within a range of from 0.1 ng/ml to 1.0 ng/ml, it was considered that the minor cancer probably occurred. Further, in the analysis of the ALP isozymes, it is possible to detect the presence of the minor cancer without fail by using: the variation of the APA; the ratio of the ALP II to the ALP III (i.e., II/III); and, a ratio of the ALP I.

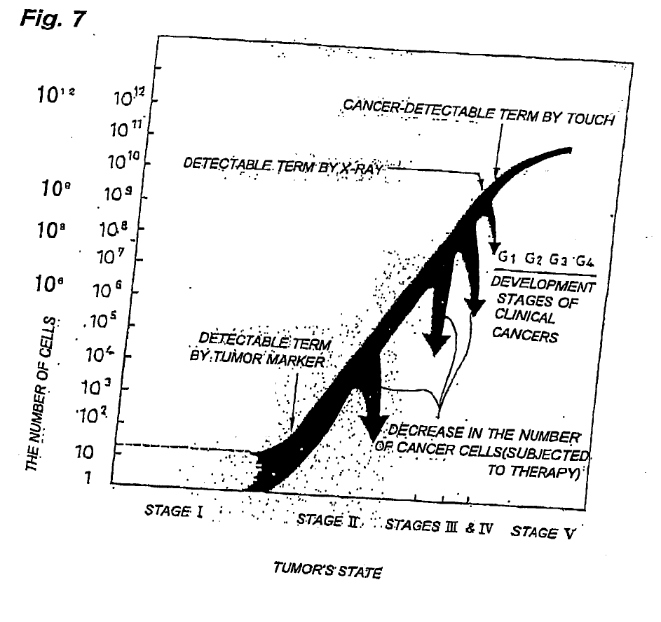

FIG. 7 shows a five-stage estimation method based on both the development level of the cancers and the analysis of the ALP isozymes.

The development level, that is, growth level of the tumors determined by screening of the tumor markers is classified into several stages in accordance with the degree of development or growth of the tumors to find out cancers occurred in a normal person in appearance. In addition to this, the risk of the cancers is estimated. For example, the above, classification defines a plurality of abnormal-level stages. In this classification: a state quite free from any minor cancer was defined as a stage I; precancer was classified into a stage II and a stage III; a pre-clinical cancerous state was defined as a stage IV; and, a state, in which a cancer having a weight of 1 Kg or more might exist, is defined as a stage V.

### INDUSTRIAL APPLICABILITY

As described above, in the method of the present invention of cancer estimation using ALP isozymes, the ALP I occurs when the total bioactivity found in the cancer cells of protopathic liver cancers, liver immersion oils, congested livers and also found in adipohepatic cells increases. This makes it possible to predict the development of each of these cancers. The ALP II is called “hepatogenous ALP”, and found also in cancerous hydropericardiums. When the total bioactivity of the ALP III increases, it is known that such total bioactivity increases in any one of the osteoglast's new generation, the hepatocirrhosis and the chronic kidney failure. There is an intimate relationship between the proliferation of the cancer cells and the patterns of the isozymes ranging from ALP I to ALP IV in a normal range of the ALP's total bioactivity in the serum. Due to this, by analyzing a change (Δ) in value of the tumor marker for a period of a predetermined time and analyzing the isozymes, it is possible to detect the presence of any minor cancer having cancer cells the number of which is from 10⁴ to 10⁹.

Even when the patterns of the ALP isozymes are not clearly identified, it is possible to precisely estimate any one of various types of cancers such as the minor cancer, clinical cancers and the like by introducing a plurality of stage-classification models, which correspond to various development stages of the cancers having been classified by using the tumor markers. As is clear from the above, the method of the present invention is excellent in effect.

1. A method of cancer estimation using ALP isozymes for finding out occurrence of a minor cancer and estimating the risk of said cancer through a process comprising the steps of:
applying a blood serum to a support element having been immersed in a buffer solution;
fractioning said support element at a predetermined liquid temperature through an electrophoresis;
isolating proteins from said serum;
color-developing said proteins with the use of an ALP isozyme-staining solution to detect said ALP isozymes; and,
estimating the risk of said cancer, characterized in that:
proliferation of cells of said cancer is totally analyzed by using values of APA and APT, wherein said values are determined with reference to patterns of ALP I to ALP V isozymes on the basis of the following parameters:
occurrence of ALP I and a ratio of said occurrence in said ALP isozymes;
a ratio of ALP II to ALP III in bioactivity in said ALP isozymes; and,
a change in ALP isozyme angle (ALP-A) in said ALP isozymes, said change showing both ALP II and ALP III in sharpness in said ALP isozymes;
whereby the proliferation state of said minor cancer is estimated.

2. The method of cancer estimation using the ALP isozymes as set forth in claim 1, wherein:
said APA obtained from said ALP isozymes is represented by the following equation:
APA=α/θ1,
where θ1 is an angle formed between: a tangential line appearing in the anode side of said ALP II; and, a line extending between a peak point of said ALP II to and a peak point of said ALP III, and said α is a distance between an intersection of these two tangential lines and said peak point of said ALP III, and
in a condition in which ALP IV occurs, said APA is represented by the following equation:
APA=β/θ2,
where β is a distance (cm) between: an intersection with said tangential line of said ALP II and a peak point of each of said ALP III and said ALP IV; and
a value of said APT is determined by dividing an overlapping line (γ cm) by a dividing line (δ cm), wherein said overlapping line (γ cm) extends from said ALP II to a change point where said ALP II changes into said ALP III; and, said dividing line (δ cm) is obtained in a position lower than said peak point of said ALP II by 1 cm.

3. The method of cancer estimation using the ALP isozymes as set forth in claim 1 or 2, wherein:
said serum is heat-treated at a temperature of 56° C.; and,
each of said patters of said ALP isozymes ranging from said ALP II to said ALP IV is reconstructed to form a reconstructed patter which is analyzed to precisely identify each of said ALP II, said ALP III and said ALP IV.

4. The method of cancer estimation using the ALP isozymes as set forth in claim 3, wherein:
the proliferation state of said cancer's cells is analyzed using changes in each of said patterns of said ALP isozymes;
a tumor is classified in its proliferation level into the following five stages by using a tumor marker:
a stage I which is an ideal condition free from any minor cancer;
a stage II corresponding to a precancerosis measured in microgram unit;
a stage III corresponding to a precancerosis measured in milligram unit; a stage IV corresponding to a so-called “precancerosis”; and,
a stage V in which the amount of cancers is equal to or more than 1 gram;
a change of said tumor marker with elapsed time is analyzed in combination with the analyzation of said cancer's cells in its proliferation state;
whereby the presence of said minor cancer and its proliferation state is judged.
